(19) United States
(12) Patent Application Publication
(10) Pub. No.: US 2005/0059718 A1
(43) Pub. Date: Mar. 17, 2005

(54) Methods of using zonisamide as an adjunctive therapy for partial seizures

(76) Inventors: Hayato Miyachi, Kanagawa (JP); Ivan Lieberburg, Berkeley, CA (US)

Correspondence Address: WILMER CUTLER PICKERING HALE AND DORR LLP, THE WILLARD OFFICE BUILDING, 1455 PENNSYLVANIA AVE, NW, WASHINGTON, DC, 20004, US

(21) Appl. No.: 10/784,145

(22) Filed: Feb. 23, 2004

Related U.S. Application Data
(60) Provisional application No. 60/448,970, filed on Feb. 21, 2003.

(51) Int. Cl. A61K 31/42

(52) U.S. Cl. 514/379

(57) ABSTRACT

Methods of using zonisamide as an adjunctive therapy for partial seizures are disclosed. In particular, the methods enhance the safety of patients taking pharmaceutical formulations of zonisamide by providing information that increases the awareness of monoclonal gammopathy of undetermined significance (MGUS), smoldering multiple myeloma (SMM), and/or multiple myeloma (MM) as possible side effects; wherein the patients and/or prescribing physicians and other medical care providers are advised to monitor for such conditions and employ methods that will improve the therapeutic outcome in the few patients who experience MGUS, SMM, and/or MM associated with zonisamide therapy.

This application claims priority to U.S. provisional application No. ______, filed ______, which is herein incorporated by reference.

### FIELD OF THE INVENTION

The present invention relates to methods of improving the safety of patients who are receiving administrations of zonisamide (3-benzisoxazole methylene sulfonamide) and those who are in need of zonisamide therapy.

### BACKGROUND OF THE INVENTION

In the United States, over 2 million serious adverse drug reactions (ADRs) occur ever year, with 100,000 associated deaths. This places ADRs as the fourth leading cause of death, ranking ahead of pulmonary disease, diabetes, AIDS, pneumonia, accidents, and automobile deaths. Compounding this problem is the fact that ADRs increase exponentially in patients who take four or more medications concurrently. See http://www.fda.gov/cder/drug/drug Reactions/default.htm.

Most drugs are approved by a Food and Drug Administration review process after an average of 1,500 patient exposures. Clinical trials involving this number of patients (both healthy volunteers and patients in need of the therapeutic effect of the drug under review) provide a statistically relevant sample of the population from which an assessment of safety and efficacy can be evaluated. However, some drugs have very rare toxicity profiles. Bromfenac, for example, causes hepatotoxicity in 1 out of 20,000 patients. For drugs with rare toxicity, more than 100,000 patients must be exposed to generate a warning signal for the adverse event. In instances where an adverse event is identified in association with a human therapeutic, government regulations require a post-approval follow-up after the drug has been taken to market.

Examples of very serious post-marketing events that have been identified in the recent past include Fen-Phen (fenfluramine-phentermine combination therapy) for weight loss and Rezulin (troglitazone) for diabetes, both of which were later removed from the market because the ADR risks outweighed the therapeutic benefits. Statistical and clinical analysis of large adverse event databases collected by post-marketing surveillance is one method by which identification of the rarer ADRs can be made. These surveillance efforts are typically administered by a pharmaceutical company marketing or maintaining the new drug dossier with the FDA. For more background on the occurrence and identification of ADRs see, for example, Lazarou, J. et al. JAMA 279(15):1200-1205 (1998), and Gurwitz, J. H. et al. Am J. Med. 109(2):87-94 (2000). For a discussion of techniques and difficulties inherent in identifying ADRs in adjunctive therapies of epileptic seizures, see French, J. Epilepsia 43(9): 951-955 (2002), which is hereby incorporated by reference in its entirety.

While Rezulin and Fen-Phen are notable for their extreme and potentially irreversible nature, other adverse drug reactions can be minimized or more easily reversed if they are recognized early, and appropriate and timely medical intervention is made. A few examples of frequently reversible adverse events are cardiac arrhythmias, liver function abnormalities, and irregularities in hematopoiesis. Thus, there remains a need for methods for identifying, detecting or treating adverse events associated with drug therapy, in a timely and informed manner.

### SUMMARY OF THE INVENTION

Unexpectedly, it has been found that zonisamide therapy in a very small percentage of patients can precipitate monoclonal gammopathy of undetermined significance (MGUS), smoldering multiple myeloma (SMM), or multiple myeloma (MM). It also has been found that by curtailing (either by removal, reduction, or tapering off) the administration of zonisamide dosing, alone or in conjunction with other concomitant medications, alleviation and minimization of this severe adverse event is possible. This is particularly the case when medical intervention to manage the disease and/or removal, reduction, or tapering off of zonisamide is instituted rapidly. The present invention may be useful at any stage of the disease as it develops from monoclonal gammopathy of undetermined significance (MGUS) to smoldering multiple myeloma (SMM) to multiple myeloma (MM). In particular, reversal of SMM early in the course of the disease is possible. This reversal is a new and unexpected finding in the medical arts, and is useful to treating and prescribing physicians in monitoring a patient receiving zonisamide therapy and in quickly recognizing and minimizing a serious side effect.

Accordingly, the present invention is directed to methods of using zonisamide for a regulatory agency approved use (e.g., as an adjunctive therapy for partial seizures). The methods improve the safety of zonisamide therapy for patients receiving administrations of the drug, or those who are in need of zonisamide therapy.

In some embodiments, the methods of using zonisamide as an adjunctive therapy for partial seizures improves the safety and health of patients taking zonisamide by increasing the awareness of the patient or patient's guardian that monoclonal gammopathy of undetermined significance (MGUS), smoldering multiple myeloma (SMM), or multiple myeloma (MM) is a possible side effect. Accordingly, a patient may be provided with a therapeutically effective amount of zonisamide, and the patient or the patient's guardian may be informed that hypercalcemia, renal insufficiency, fatigue, anemia, bone pain, spontaneous fractures, increased frequency or duration of infection, or abnormal urine color or odor are symptoms of MGUS, SMM, and/or MM that require prompt medical evaluation if such symptoms are experienced by the patient. As a result, the patient or patient's guardian can self-monitor for signs and symptoms of MGUS, SMM, and/or MM, and seek medical attention if such symptoms occur in order to obtain appropriate tests, diagnosis, and treatment. In some embodiments, the present methods reduce the risk of MGUS, SMM, and/or MM in patients receiving zonisamide therapy.

In other embodiments, the present invention provides methods of using zonisamide as an adjunctive therapy for partial seizures comprising informing a prescribing physician or other medical professional (e.g., an emergency medical worker) that MGUS, SMM, and/or MM may result from zonisamide therapy and to monitor a patient who is prescribed zonisamide as an adjunctive therapy for partial seizures for abnormal protein and protein levels in the blood and urine of the patient. The prescribing physician or other medical professional also may be advised that when hypercalcemia, renal insufficiency, fatigue, anemia, bone pain, spontaneous fractures, increased frequency or duration of infection, or abnormal urine color or odor is observed, an appropriate diagnostic be employed to determine whether MGUS, SMM, and/or MM is present. Such diagnostics may include monitoring the patient for abnormal paraproteinemia, M-spike protein in serum, Bence-Jones protein in urine, and/or depression of normal immunoglobulin levels. In addition, the prescribing physician or other medical professional may be advised to remove, reduce, or taper off the zonisamide dosing in the patient, and initiate appropriate supportive therapy for the underlying condition(s). In this manner, the present methods enable prescribing physicians and other health care professionals to recognize and minimize the risk associated with an adverse event, namely MGUS, SMM, and/or MM, which may occur in some patients who receive zonisamide therapy.

The present methods also include methods of administering zonisamide as an adjunctive therapy for partial seizures comprising providing packaging that includes a pharmaceutical formulation of zonisamide along with information providing a warning that zonisamide may cause MGUS, SMM, and/or MM in some patients and that one or more symptoms chosen from the group of hypercalcemia, renal insufficiency, fatigue, anemia, bone pain, spontaneous fractures, increased frequency or duration of infection, or abnormal urine color or odor are potentially signs of MGUS, SMM, and/or MM; and providing the packaging to a patient who has been prescribed zonisamide.

The medical information provided in any of the above described methods concerning the signs and symptoms of MGUS, SMM, and/or MM may alternatively be provided in layman's terms, so as to be better understood by patients or non-medical professionals. Those of skill in the medical art are familiar with the various layman's terms that can be used to describe the symptoms of MGUS, SMM, and/or MM.

### BRIEF DESCRIPTION OF THE DRAWINGS

FIG. 1 is a graph that plots the level of total protein over time, and the IgG level over the latter portion of the two years of the ten years of experience with the patient in Example 1. Zonegran and valproate dosages are indicated over the time span.

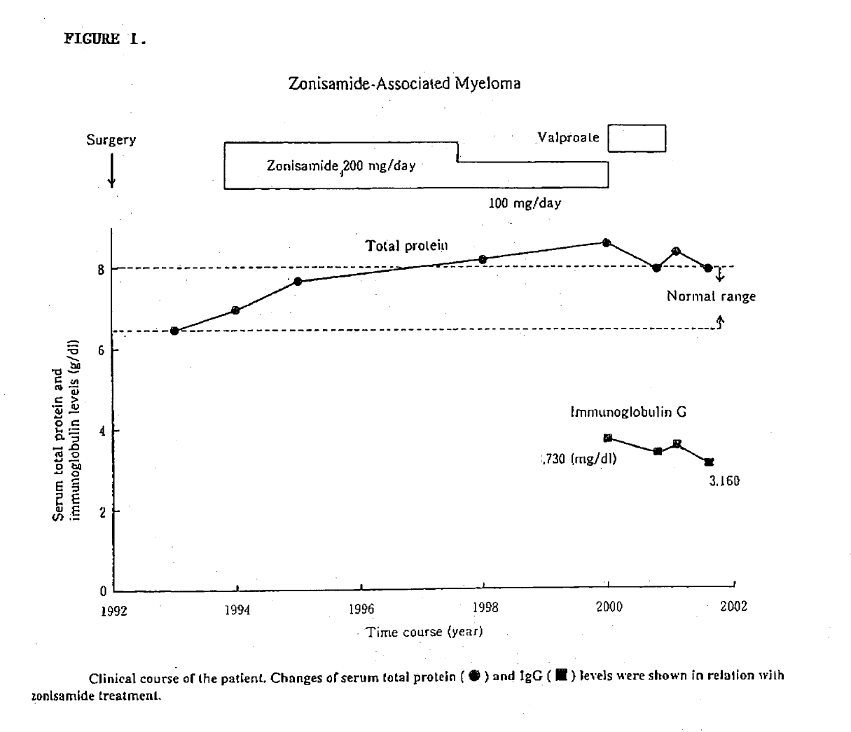

### DEFINITIONS

The following terms, while familiar to those of skill in the medical arts, are provided to facilitate an understanding of the present invention.

The term “gammopathy” refers to a primary disturbance in immunoglobulin synthesis of a patient.

“Monoclonal gammopathy” refers to any of a group of disorders that are typically associated with the proliferation of a single clone of lymphoid or plasma cells (normally visible on serum protein electrophoresis (SPEP) as a single peak) and characterized by the presence of monoclonal immunoglobulin in the serum or urine of a patient.

“Electrophoresis” is a separation technique that utilizes the movement of charged particles in an electrical field toward or away from an electric pole (anode or cathode). Thus, biomolecules may be separated and/or purified on the basis of their charge using this technique. Serum protein electrophoresis (SPEP) is a particular application of this separation technique. SPEP is effected by loading serum proteins on a gel and applying an electric field across the gel. By using a polyacrylamide gel, protein constituents are separated based on charge and molecular size.

“Multiple myeloma” refers to a malignant proliferation of plasma cells that typically originates in bone marrow, involves chiefly the skeleton of a patient, and presents clinical features attributable to the particular sites of involvement and abnormalities in formation of plasma proteins. The condition is usually characterized by numerous diffuse foci or nodular accumulations of abnormal or malignant plasma cells in the marrow of various bones (especially the skull), causing palpable swellings of the bones, and occasionally in extraskeletal sites. Upon radiological exam, the bone lesions may have a characteristic “punched out” appearance. The cells involved in the myeloma typically produce abnormal proteins and/or abnormal protein levels in the serum and urine. The disease typically develops from monoclonal gammopathy of undetermined significance (MGUS) to smoldering multiple myeloma (SMM) to multiple myeloma (MM). Symptoms of these conditions vary and are described in more detail below, but may include hypercalcemia, renal insufficiency, fatigue, anemia, bone pain, spontaneous fractures, increased frequency or duration of infection, or abnormal urine color or odor.

An “M-spike” refers to a monoclonal peak that is typically visualized as a narrow band on electrophoretic gel, or an abnormal arc in immunoelectrophoresis. It represents a proliferation of homogenous immunoglobulin produced by clone cells originating from a single common cell, e.g., a monoclonal immunoglobulin characterized by a heavy chain of a single class and subclass, and light chain of a single type (also referred to as a M-protein, a monoclonal protein, and more broadly as a paraprotein).

“Immunoelectrophoresis” refers to a lab test routinely used in the medical arts in which the components of one group of immunological reactants are first separated on the basis of electrophoresis in a gel, then identified on the basis of their precipitation with known detection agents. Typically, precipitating antibodies are used as the detection agents, which leave characteristic arcs when they are visualized after they precipitate with the detection agents in the electrophoretic gel. Serum protein immunoelectrophoresis (SPIEP) may be used in the diagnosis of MGUS and MM to identify the component light (L-) and heavy (H-) chains of a specific monoclonal protein.

“Amyloid” refers to a glycoprotein that is deposited extracellularly in tissues in amyloidosis. It is often one of the abnormal proteins detected in the blood or urine of a patient with multiple myeloma. The glycoproteins are organized in beta-pleated sheets making them relatively insoluble. As a result, the proteins form deposits in the body that can cause dysfunctions in various organs and tissues (e.g., kidney abnormalities due to obstruction of the tubules). The glycoprotein may derive from light chain of immunoglobulin (amyloid of immune origin, “AIO”), a 5-18 kD glycoprotein, typically the N terminal part of lambda or kappa light chain, produced by a single clone of plasma cells. Alternatively, the glycoprotein may be of unknown origin (amyloid of unknown origin, “AUO”), such as from serum amyloid A (SAA), one of the acute phase proteins that increases many fold during the process of inflammation.

“Monoclonal gammopathy of undetermined significance,” MGUS, or “benign monoclonal gammopathy.” refers to a condition characterized by monoclonal paraproteinemia (e.g., IgM, IgG, IgA, as evidenced by SPEP) but generally lacking other evidence of plasma cell disease. Generally, paraproteinemias are a group of diseases caused by an uncontrolled proliferation of a single clone of plasma cells giving rise to a pathological increase in monoclonal immunoglobulins. Screening tests have shown that about one to three percent of the US population have biochemical signs of gammopathy and that the majority of them are asymptomatic (Axelsson et al. 1966, Kyle et al. 1972). However, a portion of them may later show signs of a plasma cell dyscrasia such as myeloma. The paraproteins in multiple myeloma are IgG in about 50% of the cases, and IgA in about 20% of the cases, with other more rare immunoglobulins making up the balance.

### DETAILED DESCRIPTION OF THE INVENTION

Zonisamide is an antiseizure drug, chemically classified as a sulfonamide and unrelated to other antiseizure agents. Antiepileptic drugs are commonly abbreviated as “AEDs.” The active ingredient is zonisamide, 1,2-benzisoxazole-3-methanesulfonamide. Zonisamide was approved in 2000 for the adjunctive treatment, i.e., taken in conjunction with one or more other AED, treatment of epilepsy in the United States. It was first introduced in Japan approximately 12 years ago, where it also has been used as monotherapy, i.e., without other AEDs as concomitant therapeutics. Zonisamide is not known to be a hepatic enzyme inducer and has been administered adjunctively with almost all of the other regulatory-approved AEDs either in the United States or abroad.

The precise mechanism(s) by which zonisamide exerts its anti-seizure effect is unknown. Zonisamide may produce antiseizure effects through action at sodium and calcium channels. In vitro pharmacological studies suggest that zonisamide blocks sodium channels and reduces voltage-dependent, transient inward currents (T-type Ca²⁺ currents), consequently stabilizing neuronal membranes and suppressing neuronal hypersynchronization, thus suppressing hyperexcitablity in epileptic foci. In vitro binding studies have demonstrated that zonisamide binds to the GABA/benzodiazepine receptor ionophore complex in an allosteric fashion, which does not produce changes in chloride flux. Other in vitro studies have demonstrated that zonisamide (10-30 μg/mL) suppresses synaptically-driven electrical activity without affecting postsynaptic GABA or glutamate responses (cultured mouse spinal cord neurons) or neuronal or glial uptake of [³H]-GABA (rat hippocampal slices). Thus, zonisamide does not appear to potentiate the synaptic activity of GABA. In vivo microdialysis studies demonstrated that zonisamide facilitates both dopaminergic and serotonergic neurotransmission. Zonisamide also has weak carbonic anhydrase inhibiting activity (about 1/50^{th} the inhibition compared to acetazolamide), and this pharmacologic effect is not thought to be a major contributing factor in the anti-seizure activity of zonisamide.

ZONEGRAN® (the human therapeutic pharmaceutical formulation containing zonisamide) is indicated as adjunctive therapy for the treatment of partial seizures in adults and is supplied by prescription in the form of 25, 50, and 100 mg capsules. The capsule may be divided, so as to offer smaller increments in dosage. Recommended dosing is once or twice daily, the recommended daily dose of 100 mg at the initiation of therapy should not be divided. ZONEGRAN® is given orally and can be taken with or without food. While other therapeutic uses of zonisamide have been reported, such as treatment of obesity and eating disorders, treatment of neuropathic pain, prophylaxis of migraine attacks, and treatment of mania, these are not indications approved by the Food and Drug Administration (FDA) in the United States, and so are called “off-label” uses. Off-label uses, which are within the discretion of the prescribing physician to write, are also encompassed in the methods presented herein.

Prescribing physicians are informed in the product insert (which contains prescribing information approved by the FDA) that, because of the long half-life of zonisamide, up to two weeks may be required to achieve steady-state levels upon reaching a stable dose or following dosage adjustment. Although the regimen described below has been shown to be tolerated, the prescriber may wish to prolong the duration of treatment at the lower doses in order to fully assess the effects of zonisamide at steady state, noting that many of the side effects of zonisamide are more frequent at doses of 300 mg per day and above. Although there is some evidence of greater response at doses above 100-200 mg/day, the increase appears small and formal dose-response studies have not been conducted.

The initial dose should be 100 mg daily. After two weeks, the dose may be increased to 200 mg/day for at least two weeks. It can be increased to 300 mg/day and 400 mg/day, with the dose stable for at least two weeks to achieve steady state at each level. Evidence from controlled trials suggests that ZONEGRAN® doses of 100-600 mg/day are effective, but there is no suggestion of increasing response above 400 mg/day.

Adjunctive therapy for partial seizures in adults denotes that these patients are already on other anti-epileptic medications, but that they are continuing to seize at a rate that has been deemed by their treating physician to require additional (add-on) therapy. For a recent review of AEDs currently available to American physicians, their efficacies for particular types of epileptic seizures and associated ADRs, see: Ilo Leppik, Epilepsia 42(Suppl.4): 1-6 (2001).

The use of multiple anti-epileptic medications in the adjunctive setting increases the likelihood of confluent or interactive ADRs, and also may confuse the treating physician as to the causal agent. For instance, when an attending medical professional is presented with a patient taking a combination of medications and manifesting a particular side-effect, it is difficult to diagnose which of the patient's medications (or combination of medications) is responsible for the observed side effect. Typically, the attending physician must consult the medical literature of known adverse events to identify drug(s) that are most likely to cause the observed side-effects. Known adverse events may also be found in the package drug inserts for each drug. The drug(s) having the higher likelihood of causing the observed side-effects are usually reduced or withdrawn first. When such options are exhausted, the patient may have to be systematically withdrawn from the various drugs until the cause is identified. Since zonisamide is typically prescribed as an adjunctive therapy, it presents such complications when side-effects occur.

This situation is further complicated when side-effects occur that are not normally associated with a particular drug. For example, phenyloin (Dilantin®) therapy has been implicated in several incidents of multiple myeloma, including resultant fatalities. See, for example, Kanoh, T., et al., Rinsho Ketsueki, 37(3): 239-43 (1996). Carbamazepine, phenobarbital and primidone have also been associated with development of MM. In contrast, zonisamide has not been known to cause MGUS, SMM, and/or MM in patients receiving ZONEGRAN® therapy. In fact, in a study of immunoglobulin levels in patients on regular dosages of zonisamide, there was no significant correlation between the dosage or serum level of zonisamide and immunoglobulin levels in the patients. Fujimoto, Y., Arneim. Forsch., 40 (II) nr. 8, pp. 855-858 (1990).

Given this knowledge of adverse events, a medical professional would not suspect zonisamide to be responsible for causing MGUS, SMM, and/or MM in a patient exhibiting the relevant symptoms. Consequently, the attending medical professional would have no obvious reason to withdraw such a patient from zonisamide, and would allow the therapy to continue while searching for other sources of the condition. However, a careful review of the data generated in American clinical trials, as well as in ADR reports gathered once commercial marketing began, has yielded the discovery that zonisamide may independently induce MGUS, SMM, and/or MM in a small number of patients, and has implicated this condition in patients receiving zonisamide as an adjunctive therapy. Accordingly, the present invention is directed to methods of increasing the safety of zonisamide therapy in view of its newly discovered role in MGUS, SMM, and/or MM.

Multiple myeloma (MM) is typically recognized clinically by the proliferation of malignant plasma cells in the bone marrow of a patient. These neoplastic plasma cells produce immunoglobulins and evolve from B-lymphocytes. The immunoglobulins that are produced by the plasma cells may be detected in the blood serum and/or urine of a patient by electrophoresis testing. Clinical symptoms include anemia, hypercalcemia, renal insufficiency, and lytic bone lesions. Distinctions in the course and the severity of the disease as it develops from monoclonal gammopathy of undetermined significance (MGUS) to smoldering multiple myeloma (SMM) to multiple myeloma (MM) are provided in Table I and Table II below. The tables also summarize methods of detection, diagnosis, and monitoring of these conditions. Such symptoms and techniques are familiar to those of skill in the art.

TABLE 1
Comparison of Clinical Features of MM, SMM, or MGUS

| Characteristic | MM | SMM | MGUS |
|---|---|---|---|
| Marrow plasma | >=10% | >=10% | <10% |
| cells | | | |
| Serum M-spike | >=3 g/dL | >=3 g/dL | <3 g/dL |
| Bence-Jones | >=1 g/24 h | <1 g/24 h | <1 g/24 h |
| protein in urine | | | |
| Anemia | usually present | may be present | absent |
| Hypercalcemia, | may be present | absent | absent |
| renal insufficiency | | | |
| Lytic bone lesions | usually present | absent | absent |

MM = multiple myeloma
SMM = smoldering multiple myeloma
MGUS = monoclonal gammopathy of undetermined significance

TABLE II
Classifying stages by severity and clinical features of
multiple myeloma

Stages of disease progression
Stage I
Relatively few cancer cells have spread throughout the body. The
number of red blood cells and the amount of calcium in the blood are
normal. No tumors (plasmacytomas) are found in the bone. The
amount of M-protein in the blood or urine is very low. There may be
no symptoms of disease.
Stage II
A moderate number of cancer cells have spread throughout the
body.
Stage III
A relatively large number of cancer cells have spread throughout the
body. There may be one or more of the following:
A decrease in the number of red blood cells, causing anemia.
The amount of calcium in the blood is very high, because the
bones are being damaged.
More than three bone tumors (plasmacytomas) are found.
High levels of M-protein are found in the blood or urine.
Clinical features of MM
Hypercalcemia
Renal insufficiency
Anemia
Lytic bone lesions (skeletal survey):
Pain
Spontaneous fractures; compression fractures
Monoclonal protein:
SPEP (serum protein electrophoresis)
SPIEP (serum protein immunoelectrophoresis)
Urine protein immunoelectrophoresis (Bence - Jones protein)
Depression of normal lg's:
increased infection risk
Diagnosis of MM
>10% plasma cells in marrow or aggregates on biopsy or a
plasmacytoma
Monoclonal protein:
Serum M-protein >3 g/dl or
M-protein in urine
Plus one or more of the following:
Anemia
Hypercalcemia

If a patient develops MM, SMM, or MGUS while on zonisamide therapy, the treating physician should search for other causes of that condition. Should no other obvious causes be identified, zonisamide should ordinarily be removed, reduced, or alternatively tapered down to an acceptable level, and alternative treatment for the underlying medical condition may be initiated as clinically indicated. If another cause for the attack is identified then it may be possible to carefully re-challenge the patient with zonisamide once the MM, SMM, or MGUS has subsided. If the patient again appears to be developing MM, SMM, or MGUS, or is diagnosed with one of these conditions, then switching to another AED may be warranted.

In patients experiencing hypercalcemia, renal insufficiency, fatigue, anemia, bone pain, spontaneous fractures, increased frequency or duration of infection, or abnormal urine color or odor, or in instances where MM, SMM, or MGUS is suspected, an appropriate diagnostic laboratory test should be performed in accordance with the those outlined in Tables 1 and 2, above. The diagnostic may include a test for paraproteinemia, M-spike protein in serum, Bence-Jones protein in urine, or depression of normal immunoglobulin levels of the patient; if these tests reveal an abnormality, and no other cause is obvious, then the drug should typically be withdrawn or titrated down to a level where the side-effect is no longer a concern. The diagnostic tests may be repeated, as needed, to monitor the patient until the symptoms subside.

In some cases, it may be possible to reduce or taper-off the level of zonisamide to avoid MM, SMM, MGUS, or other side-effects, while maintaining the therapeutic efficacy of the drug therapy. Such decisions may be made by an attending medical personnel, for example, after considering the severity of the MM, SMM, MGUS, or other side effects in relation to the patient's need for continued zonisamide therapy.

Other complications must be treated as they arise, and a skilled physician of emergency or internal medicine knows such treatments. For example, abruptly removing anti-epileptic drug therapy from an epileptic patient may result in more severe or more frequent seizures or status epilepticus. Therefore, removal of zonisamide therapy carries the risk of more severe seizures. However, a hospital physician or emergency medical personnel will have access to other pharmacological interventions for short-term control of generalized seizure activity such as either intravenous lorazepam, at a dose of 0.1 mg/kg, or diazepam at 0.2 mg/kg. If sedatives prove insufficient, then a patient also may be administered fosphenyloin, or in status epilepticus, phenobarbital, with careful monitoring for respiratory depression. Intravenous administration is preferred since this route will provide the most rapid attainment of therapeutic serum levels. Additionally, at the treating physician's discretion, an alternate AED may be substituted for zonisamide.

### EXAMPLE 1

A 39-year old man was found to have hyperproteinemia (8.6 g/dl). He had neither personal history, nor apparent family history, of exposure to radiation or chemotherapy. Ten years prior he had had surgical treatment of a subarachnoid hemorrhage due to an arteriovenous malformation. As a result of the brain injury caused by the hemorrhage and/or reparative surgery, he has developed generalized seizures which had been treated with the anticonvulsant drug zonisamide at a daily dose of 200 mg/day for about 5 years followed by a dosage reduction to 100 mg/day. He had been taking no other drug during this ten-year period.

By review of his medical record, the level of serum total protein had gradually increased from 6.5 g/dl at year 3 of treatment to 8.2 g/dl in year 8. The patient presented with no complaint of bone pain. Laboratory examination showed an elevated serum level of immunoglobulin G IgG (3.68 g/dl) with suppressed levels of IgM 38 mg/dl) and IgA (40 mg/dl). Protein fractionation showed an M-peak. Immunoelectrophoresis fractionation of the serum protein revealed M-protein composed of IgG with a single lambda type of L-chain. Bence-Jones protein was not demonstrated in the urine. Serum levels of creatinine, calcium and beta2-microglobulin were not elevated. Peripheral blood examination showed no cytopenia. Helper T cell count (CD4) was normal, X-ray findings of the skull showed equivocal bone lesions reflecting a history of brain surgery. X-ray findings of ribs, lumbar spine and pelvis were within the normal limit. Bone marrow aspiration revealed moderately increased plasma cells in 8.1% of the nucleated cells. Chromosomal analysis of the bone marrow showed a normal karyotype of 46 XY. Electroencephalogram showed irregular spikes and waves, which were dominant in the right fronto-parietal area indicating that there was still epileptic discharge in the brain.

Zonisamide was replaced by sodium valproate for treatment of the seizures. This new medication regimen prevented convulsions and there was no significant increase in the serum concentration of IgG during the 13-month observation follow-up period of the patient.

The clinical manifestations of myeloma vary from smoldering myeloma to symptomatic plasma cell dyscrasia. A lot of variables, alone or in combination, have been used as a discriminating index of malignant proliferation of B-lymphocytes or plasma cells. In this patient, the clinical features of malignant B-lymphocyte or plasma cell disorder were absent, including osteolysis, suppression of hemopoiesis, hypercalcemia and renal dysfunction. However, a moderately increased number of marrow plasma cells (>5%) and a high concentration of M-protein (>3.5 g/dl) with suppressed levels of other classes of immunoglobulin suggested a monoclonal malignant proliferation of B-lymphocyte, that lead to a diagnosis of smoldering multiple myeloma.

Multiple myeloma is prevalent in the elderly, usually over the age of 60 years, and can also occur in younger adults, but rarely does onset occur before the age of 40. Since the patient was in this case much younger than the typical age for multiple myeloma, the presence of extrinsic etiological factors was considered. Other anticonvulsants have been suggested to have a causal association with multiple myeloma, and have been associated with M-protein spikes of type IgG (lambda). Other AEDs associated with MM occurrence include phenyloin, carbamazepine, phenobarbital and primidone (of these only the former two drugs are marketed as AEDs in the United States). These four drugs are also known enzyme-inducing antiepileptic drugs (EIAEDs), that is, they induce higher levels of the liver enzymes that are involved ion their metabolism. Zonisamide has no liver enzyme inducing activity and also lacks the urea functional group that is common to these AEDs.

Since a considerable proportion of patients with smoldering myeloma progress to symptomatic myeloma, the findings in this patient are surprising and the long-term reversibility of the disease in this patient is still under investigation. Given the seriousness of development of multiple myeloma, the early detection and reversal of M-protein spike and immunoglobulin deficiency early provides a physician early notice to substitute a different anticonvulsant therapy that may stop, slow, or reverse the progression to malignancy.

1. A method of using zonisamide as an adjunctive therapy for partial seizures to improve the safety of such therapy comprising:
providing a patient with a therapeutically effective amount of zonisamide, and
informing the patient or the patient's guardian during the course of zonisamide therapy that hypercalcemia, renal insufficiency, fatigue, anemia, bone pain, spontaneous fractures, increased frequency or duration of infection, or abnormal urine color or odor are symptoms of monoclonal gammopathy of undetermined significance (MGUS), smoldering multiple myeloma (SMM), or multiple myeloma (MM) that require prompt medical evaluation if such symptoms are experienced by the patient.

2. The method of claim 1 wherein the therapeutically effective amount of zonisamide is from 25 mg to 600 mg.

3. The method of claim 1 wherein the therapeutically effective amount of zonisamide is provided in unit dose form.

4. The method of claim 1 wherein the therapeutically effective amount of zonisamide is provided in a unit dose form and in multiple doses to provide for a course of therapy.

5. The method of claim 4, wherein the unit dose is from 25 mg to 200 mg.

6. A method of using zonisamide as an adjunctive therapy for partial seizures to improve the health of a patient receiving such therapy comprising: providing a patient with a therapeutically effective amount of zonisamide, and
informing the patient or the patient's guardian during the course of such therapy that hypercalcemia, renal insufficiency, fatigue, anemia, bone pain, spontaneous fractures, increased frequency or duration of infection, or abnormal urine color or odor are symptoms of monoclonal gammopathy of undetermined significance (MGUS), smoldering multiple myeloma (SMM), or multiple myeloma (MM) that require prompt medical evaluation if such symptoms are experienced by the patient.

7. The method of claim 6 wherein the therapeutically effective amount of zonisamide is from 25 mg to 600 mg.

8. The method of claim 6 wherein the therapeutically effective amount of zonisamide is provided in unit dose form.

9. The method of claim 6 wherein the therapeutically effective amount of zonisamide is provided in a unit dose form and in multiple doses to provide for a course of therapy.

10. The method of claim 9, wherein the unit dose is from 25 mg to 200 mg.

11. A method of using zonisamide as an adjunctive therapy for partial seizures to reduce the risk of monoclonal gammopathy of undetermined significance (MGUS), smoldering multiple myeloma (SMM), or multiple myeloma (MM) in a patient receiving such therapy comprising:
providing the patient with a therapeutically effective amount of zonisamide, and
informing the patient or the patient's guardian during the course of zonisamide therapy that hypercalcemia, renal insufficiency, fatigue, anemia, bone pain, spontaneous fractures, increased frequency or duration of infection, or abnormal urine color or odor are symptoms of monoclonal gammopathy of undetermined significance (MGUS), smoldering multiple myeloma (SMM), or multiple myeloma (MM) that require prompt medical evaluation if such symptoms are experienced by the patient.

12. The method of claim 11 wherein the therapeutically effective amount of zonisamide is from 25 mg to 600 mg.

13. The method of claim 11 wherein the therapeutically effective amount of zonisamide is provided in unit dose form.

14. The method of claim 11 wherein the therapeutically effective amount of zonisamide is provided in a unit dose form and in multiple doses to provide for a course of therapy.

15. The method of claim 14, wherein the unit dose is from 25 mg to 200 mg.

16. A method of using zonisamide as an adjunctive therapy for partial seizures comprising:
enhancing the safety profile of zonisamide by informing a prescribing physician that monoclonal gammopathy of undetermined significance (MGUS), smoldering multiple myeloma (SMM), or multiple myeloma (MM) may result from zonisamide therapy;
advising the physician to monitor a patient who is prescribed zonisamide as an adjunctive therapy for partial seizures for one or more symptoms chosen from the group of hypercalcemia, renal insufficiency, fatigue, anemia, bone pain, spontaneous fractures, increased frequency or duration of infection, or abnormal urine color or odor, and when such a symptom is observed,
recommending that a laboratory test for paraproteinemia, M-spike protein in serum, Bence-Jones protein in urine, or depression of normal immunoglobulin levels be performed, and
if the test reveals an abnormal result for that patient, recommending that the physician consider removing, reducing, or tapering off zonisamide dosing in the patient while initiating appropriate supportive therapy.

17. A method of using zonisamide as an adjunctive therapy for partial seizures comprising:
improving patient outcome by informing an emergency medical worker that a patient who is receiving zonisamide as an adjunctive therapy for partial seizures and exhibits hypercalcemia, renal insufficiency, fatigue, anemia, bone pain, spontaneous fractures, increased frequency or duration of infection, or abnormal urine color or odor, may be suffering from monoclonal gammopathy of undetermined significance (MGUS), smoldering multiple myeloma (SMM), or multiple myeloma (MM); and when such a symptom is observed,
recommending that an appropriate laboratory test for paraproteinemia, M-spike protein in serum, Bence-Jones protein in urine, or depression of normal immunoglobulin levels be performed,
and if the result of that test is abnormal for that patient, recommending that the worker initiate appropriate supportive therapy and discontinue or reduce zonisamide dosing in the patient.

18. The method of any of claim 17 wherein the diagnostic comprises measurement of M-spike protein in serum.

19. The method of claim 17 wherein the diagnostic comprises measurement of Bence-Jones protein in urine.

20. The method of any of claims 17 wherein the prescribed dosage of zonisamide is from 25 mg to 600 mg.

21. The method of claim 17 wherein the therapeutically effective amount of zonisamide is provided in unit dose form.

22. The method of claim 17 wherein the patient is receiving zonisamide in a therapeutically effective amount provided in a unit dose form and in multiple doses to provide for a course of therapy.

23. The method of claim 21 wherein the unit dose is from 25 mg to 200 mg.

24. A method of using zonisamide as an adjunctive therapy for partial seizures comprising:
providing packaging that includes a pharmaceutical formulation of zonisamide along with information providing a warning that zonisamide may cause monoclonal gammopathy of undetermined significance (MGUS), smoldering multiple myeloma (SMM), or multiple myeloma (MM) in some patients and that one or more symptoms chosen from the group of hypercalcemia, renal insufficiency, fatigue, anemia, bone pain, spontaneous fractures, increased frequency or duration of infection, or abnormal urine color or odor are potentially signs of monoclonal gammopathy of undetermined significance (MGUS), smoldering multiple myeloma (SMM), or multiple myeloma (MM); and
providing such packaging to a patient who has been prescribed zonisamide.

25. The method of claim 24 wherein the formulation contains a therapeutically effective amount of zonisamide of from 25 mg to 600 mg.

26. The method of claim 24 wherein the therapeutically effective amount of zonisamide is provided in unit dose form.

27. The method of claim 24 wherein the therapeutically effective amount of zonisamide is provided in unit dose form and in multiple doses to provide for a course of therapy.

28. The method of claim 24 wherein the unit dose is from 25 mg to 200 mg.

29. A method of using zonisamide as an adjunctive therapy for partial seizures comprising:
providing a patient with a therapeutically effective amount of zonisamide and a therapeutically effective amount of at least one other anti-epilepsy drug, and
informing the patient or the patient's guardian that hypercalcemia, renal insufficiency, fatigue, anemia, bone pain, spontaneous fractures, increased frequency or duration of infection, or abnormal urine color or odor are symptoms of monoclonal gammopathy of undetermined significance (MGUS), smoldering multiple myeloma (SMM), or multiple myeloma (MM) that require prompt medical evaluation if such symptoms are experienced by the patient.

30. The method of claim 29, wherein the patient or patient's guardian is informed by reference to a package drug insert.

31. A method of administering zonisamide as an adjunctive therapy for partial seizures comprising:
providing a patient with a therapeutically effective amount of zonisamide and a therapeutically effective amount of at least one other anti-epilepsy drug; and
informing the patient or the patient's guardian that hypercalcemia, renal insufficiency, fatigue, anemia, bone pain, spontaneous fractures, increased frequency or duration of infection, or abnormal urine color or odor are symptoms of monoclonal gammopathy of undetermined significance (MGUS), smoldering multiple myeloma (SMM), or multiple myeloma (MM) that require prompt medical evaluation if such symptoms are experienced by the patient.

32. The method of claim 31, wherein the patient or patient's guardian is informed by reference to a package drug insert.

33. A method of using zonisamide as an adjunctive therapy for partial seizures comprising:
monitoring a patient who is receiving administrations of zonisamide for one or more symptoms chosen from the group of hypercalcemia, renal insufficiency, fatigue, anemia, bone pain, spontaneous fractures, increased frequency or duration of infection, or abnormal urine color or odor;
if one or more of said symptoms are observed, performing a laboratory test for paraproteinemia, M-spike protein in serum, Bence-Jones protein in urine, or depression of normal immunoglobulin levels of the patient; and
if the laboratory test indicates an abnormal result for that patient, reducing or tapering off the zonisamide dosing.

34. The method of claim 33, wherein the zonisamide dosing is increased after subsequent laboratory test are determined to be normal for that patient.
